(19) United States
(12) Patent Application Publication
(10) Pub. No.: US 2022/0304909 A1
(43) Pub. Date: Sep. 29, 2022

(54) ORAL CARE COMPOSITION

(71) Applicant: Church & Dwight Co., Inc., Princeton, NJ (US)

(72) Inventors: Alysha Moretti, Feasterville, PA (US); Jennifer Benson, Newtown, PA (US)

(73) Assignee: Church & Dwight Co., Inc., Princeton, NJ (US)

(21) Appl. No.: 17/290,113

(22) PCT Filed: Nov. 1, 2019
(86) PCT No.: PCT/IB2019/059415

§ 371 Date: Apr. 29, 2021

Related U.S. Application Data
(60) Provisional application No. 62/754,956, filed on Nov. 2, 2018.

(51) Int. Cl. A61K 8/41; A61Q 11/00; A61K 8/39; A61K 8/29

(52) U.S. Cl. CPC A61K 8/416; A61Q 11/00; A61K 8/39; A61K 8/29; A61K 2800/30

(57) ABSTRACT

The present disclosure provides oral care compositions that include a water-miscible base component, an opacifying agent, a structuring agent, and an active antiseptic agent effective for providing antiseptic activity, wherein the oral care composition is opaque and exhibits antiseptic activity.

### FIELD OF THE INVENTION

The present disclosure relates to oral compositions useful for providing antiseptic activity. More particularly, the disclosure relates to stable, opaque formulations that contain an active antiseptic ingredient and that optionally is in the form of a cream.

### BACKGROUND

This disclosure relates to a stable, opaque formula that contains an active antiseptic ingredient. Current oral care formulations on the market, including private label brands and the Orajel™ brand in particular, are petrolatum-based formulations and contain a maximum of three active ingredients: benzocaine, menthol, zinc chloride, and/or benzalkonium chloride. Although some of these formulations have the appearance and consistency of a cream, these petrolatum-based formulations have been found to provide negligible antiseptic activity, even if supplemented with an antiseptic ingredient, such as benzalkonium chloride.

There are some current formulations on the market which are PEG-based formulations containing benzalkonium chloride which do demonstrate antiseptic activity. However, these PEG-based formulations on the market are in the form of a gel.

Accordingly, there is still a desire and a need to provide an oral care composition in the form of a cream that contains an active antiseptic ingredient and exhibits overall antiseptic properties. In particular, there is a need for an oral care composition that includes an opacifying agent and allows for activity of an antiseptic active ingredient to kill bacteria associated with oral infections. Additional active ingredients in the oral care composition can also provide relief of pain and various oral discomfort ailments including toothaches and mouth sores.

### SUMMARY

In one or more embodiments, the present disclosure provides an oral composition with a water-miscible base and which includes an antiseptic active ingredient and an opacifying agent, wherein the oral composition is effective to kill bacteria associated with oral infections. Current solutions for the topical treatment of bacteria associated with oral infections are mainly delivered in the form of liquids or gels, which can often dissolve, spread easily, or get dislocated and cause numbness throughout the oral mucosa. The present disclosure is directed toward formulations which enable antiseptic activity and are preferably in the form of a cream, which has been found to be preferred by certain users over gel formulas. Various embodiments of the oral care compositions described herein further include additional active ingredients which can provide relief from oral discomfort, such as toothaches and mouth sores. Other aspects and advantages of the present disclosure will become apparent from the following.

The invention includes, without limitation, the following embodiments.

Embodiment 1: An oral care composition comprising: a water-miscible base component;

an opacifying agent; a structuring agent; and an active antiseptic agent effective for providing antiseptic activity; wherein the oral care composition is opaque and exhibits antiseptic activity.

Embodiment 2: The oral care composition of any preceding embodiment, wherein the oral care composition is substantially free of petrolatum.

Embodiment 3: The oral care composition of any preceding embodiment, wherein the oral care composition comprises petrolatum in an amount of less than 20% by weight, based on the total weight of the oral care composition.

Embodiment 4: The oral care composition of any preceding embodiment, wherein the water-miscible base comprises polyethylene glycol-8 (PEG-8).

Embodiment 5: The oral care composition of any preceding embodiment, wherein the water-miscible base comprises polyethylene glycol-8 (PEG-8), and wherein the PEG-8 is present in an amount of about 30 to about 50 wt. %, based on the total weight of the oral care composition.

Embodiment 6: The oral care composition of any preceding embodiment, wherein the opacifying agent is titanium dioxide.

Embodiment 7: The oral care composition of any preceding embodiment, wherein the opacifying agent is present in an amount of about 0.05 to about 0.5 wt. %, based on the total weight of the oral care composition.

Embodiment 8: The oral care composition of any preceding embodiment, wherein the structuring agent is selected from the group consisting of PEG/PPG-116/66 copolymer, poloxomer 407, PEG-75, and combinations thereof.

Embodiment 9: The oral care composition of any preceding embodiment, wherein the structuring agent is not negatively charged.

Embodiment 10: The oral care composition of any preceding embodiment, wherein the structuring agent is present in an amount of about 20 to about 50 wt. %, based on the total weight of the oral care composition.

Embodiment 11: The oral care composition of any preceding embodiment, wherein the active antiseptic agent comprises benzalkonium chloride.

Embodiment 12: The oral care composition of any preceding embodiment, wherein the active antiseptic agent comprises benzalkonium chloride, and wherein the benzalkonium chloride is present in an amount of less than about 1 wt. %, based on the total weight of the oral care composition.

Embodiment 13: The oral care composition of any preceding embodiment, wherein the oral care composition is in the form of a cream.

Embodiment 14: The oral care composition of any preceding embodiment, further comprising one or more additional active ingredients.

Embodiment 15: The oral care composition of any preceding embodiment, further comprising one or more additional active ingredients, wherein the one or more additional active ingredients are selected from the group consisting of benzocaine, menthol, zinc chloride, and combinations thereof.

Embodiment 16: The oral care composition of any preceding embodiment, further comprising a flavoring agent.

Embodiment 17: The oral care composition of any preceding embodiment, further comprising a flavoring agent, wherein the flavoring agent is present in an amount in the range of about 0.1 to about 2 weight percent, based on the total weight of the oral care composition.

Embodiment 18: The oral care composition of any preceding embodiment, wherein the antiseptic activity exhibited by the oral care composition is defined in that the oral care composition is effective to cause at least a 99% reduction in bacteria counts for C. albicans 18804 organisms, S. mutans 25175 organisms, and A. oris 19246 organisms after contact between the oral care composition and the organisms at a temperature of about 37° C. for a time of about 10 mins

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### DETAILED DESCRIPTION

The present disclosure now will be described more fully hereinafter. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

In one or more aspects, the present disclosure provides oral care compositions comprising a water-miscible base component, one or more opacifying agents, one or more structuring agents, and one or more active antiseptic agents effective for providing antiseptic activity. As discussed in more detail below, the oral care compositions described herein are stable, opaque compositions capable of exhibiting antiseptic activity. As used herein, a stable composition is defined as a composition that does not exhibit substantial separation or, preferably, any separation when exposed to a temperature of about 40° C. and a 75% relative humidity for a time of three months.

In various embodiments of the present disclosure, the water-miscible base component of the oral compositions described herein can comprise polyethylene glycol (PEG), glycerin, propylene glycol, sorbitol, and combinations thereof. For example, in some embodiments the base component comprises polyethylene glycol (PEG). In certain embodiments, the base component comprises PEG-8.

In certain embodiments, the water-miscible base component can be present in amounts in the range of about 20 to about 90 weight percent, about 25 to about 60 weight percent, about 30 to about 50 weight percent, or about 35 to about 45 weight percent, based on the total weight of the oral care composition. In certain embodiments, the base component can be present in an amount of at least about 15 weight percent, at least about 20 weight percent, at least about 30 weight percent, at least about 40 weight percent, or at least about 50 weight percent, based on the total weight of the oral care composition (e.g., with a maximum concentration of about 90 percent by weight).

Various embodiments of the oral care compositions described herein include one or more opacifying agents. Preferably, the one or more opacifying agents include materials that are effective for configuring the formulation to be in the form of a cream. In some embodiments, the one or more opacifying agents can be selected from the group consisting of glycol distearate, cetyl alcohol, cetostearyl alcohol, zincoxide, calcium carbonate, silicas, mica, kaolin, myristic acid, stearyl alcohol, glycerol monostearate, titanium dioxide, and combinations thereof. In certain embodiments, the one or more opacifying agents is titanium dioxide.

Depending on the type of opacifying agent(s) used in the oral care compositions described herein, the weight percentage of the opacifying agent(s) in the formulation can vary. For example, pigments (e.g., titanium dioxide, silicas, mica, zincoxide, etc.) can be present in a relatively lower weight percentage. In certain embodiments, the one or more opacifying agents can be present in amounts in the range of about 0.01 to about 2 weight percent, about 0.05 to about 1 weight percent, about 0.05 to about 0.5 weight percent, or about 0.1 to about 0.3 weight percent, based on the total weight of the oral care composition. In certain embodiments, the one or more opacifying agents can be present in an amount of at least about 0.05 weight percent, at least about 0.1 weight percent, or at least about 0.2 weight percent, based on the total weight of the oral care composition (e.g., with a maximum concentration of about 2 percent by weight). In certain embodiments, the one or more opacifying agents can be present in an amount of about 1 weight percent or less, about 0.5 weight percent or less, or about 0.2 weight percent or less, based on the total weight of the oral care composition.

In some embodiments, esters, alcohols, and polymers which are useful as opacifying agents (e.g., glycol stearate, glycol distearate, cetyl alcohol, etc.) can be present in a relatively higher weight percentage. In certain embodiments, the one or more opacifying agents can each be present in amounts in the range of about 0.01 to about 20 weight percent, about 0.1 to about 10 weight percent, or about 1 to about 5 weight percent, based on the total weight of the oral care composition. In certain embodiments, the one or more opacifying agents can each be present in an amount of at least about 0.01 weight percent, at least about 1 weight percent, at least about 5 weight percent, or at least about 10 weight percent, based on the total weight of the oral care composition (e.g., with a maximum concentration of about 20 percent by weight). In certain embodiments, the one or more opacifying agents can be each present in an amount of about 20 weight percent or less, about 10 weight percent or less, or about 5 weight percent or less, based on the total weight of the oral care composition.

In various embodiments, the oral care composition comprises one or more structuring agents. The one or more structuring agents can help in stabilizing the formulation which includes an opacifying agent. In other words, a structuring agent can help prevent separation of the cream during its shelf life. The one or more structuring agents can further help to the slow dissolution of the oral care composition in the oral cavity, thereby increasing the duration of pain relief provided to the user and the effectiveness of the antiseptic activity. Structuring agents can provide a thickening effect and provide firmness to the oral care composition.

In some embodiments, the oral care composition can include only a limited amount of a structuring agent, can be substantially free of one or more specific structuring agents, or can be completely free of one or more specific structuring agents. In some embodiments, the oral care composition comprises petrolatum in an amount of less than 20% by weight, less than 15% by weight, less than 10% by weight, less than 5% by weight, or less than 1% by weight based on the total weight of the oral care composition. In some embodiments, the oral care composition can be substantially free of petrolatum (e.g., comprising less than 0.1% by weight based on the total weight of the composition) or can be completely free of petrolatum.

In various embodiments, preferred structuring agents can exhibit a defined polarity. For example, a structuring agent utilized in the present composition (or each of the one or more structuring agents utilized in the present composition) can be characterized in that they are not negatively charged. Preferably, the absence of any negative charge can increase compatibility with the antiseptic active ingredient in the oral care compositions disclosed herein. In some embodiments the one or more structuring agents is selected from the group consisting of PEG/PPG-116/66 copolymer, poloxomer 407, PEG-75, and combinations thereof. In certain embodiments, the oral care composition can further include small amounts of alternative structuring agents known in the art.

In certain embodiments, the one or more structuring agents can be present in amounts in the range of about 15 to about 50 weight percent, about 20 to about 40 weight percent, or about 25 to about 35 weight percent, based on the total weight of the oral care composition. In certain embodiments, the one or more structuring agents can be present in an amount of at least about 15 weight percent, at least about 20 weight percent, or at least about 25 weight percent, based on the total weight of the oral care composition (e.g., with a maximum concentration of about 60 percent by weight).

The compositions described herein are preferably effective to provide antiseptic activity while also exhibiting a cream-like appearance that consumers often desire. The oral compositions described herein thus can include one or more materials or compounds that are active antiseptic agents and thus are effective to provide antiseptic activity. As described in more detail in the Examples provided below, an active antiseptic agent allows for the oral care composition to exhibit at least a 99% reduction in bacteria counts for C. albicans 18804 organisms, S. mutans 25175 organisms, and A. oris 19246 organisms after subjecting the oral care composition to a temperature of about 37° C. for 10 mins.

The compositions described herein can comprise one or more antiseptic agents. In some embodiments, the antiseptic agent can be selected from the group consisting of benzethonium chloride, benzalkonium chloride, carbamide peroxide in anhydrous glycerin, cetalkonium chloride, cetylpyridinium chloride, chlorophyllin copper complex, dequalinium chloride, domiphen bromide,eucalyptol, gentian violet, hydrogen peroxide, iodine, menthol, methyl salicylate, nitromerso, oxyquinoline, phenol preparations, povidone-iodine, secondary amyltricresois, sodium caprylate, thymol preparations, tolu balsam, and combinations thereof. In various embodiments, the one or more active antiseptic agents in the oral care compositions described herein comprise benzalkonium chloride.

The one or more active antiseptic agents can be present in an amount of, for example, about 0.01 to about 3 weight percent, or about 0.05 to about 1 weight percent, or about 0.1 to about 0.5 weight percent based on the total weight of the oral care composition. In various embodiments, the antiseptic agent can be present in an amount of less than about 1 weight percent, less than about 0.5 weight percent, or less than about 0.2 weight percent, based on the total weight of the oral care composition.

In various embodiments, one or more additional active ingredients can be included in the oral care composition to provide pain relief and/or soothing and cooling effects of the product. The one or more additional active ingredients can be present in an amount suitable for providing oral pain relief. For example, the oral care compositions described herein can include an antiseptic agent (e.g., benzalkonium chloride) in combination with one active ingredient for pain relief (e.g., benzocaine) or more than one active ingredient for pain relief (e.g., benzocaine and menthol). In certain embodiment, the one or more additional active ingredients can include benzocaine, menthol, zinc chloride, or combinations thereof. Other active ingredients known in the art can be added to the oral care product as desired. Moreover, the total amount of active ingredient(s) present in the oral care composition can vary based on the type of active ingredient(s) used and/or the number of different active ingredients used.

For example, menthol can be used in the oral care composition to provide additional pain relief specifically to the gums via the cooling effect of the active ingredient. Menthol can be present in an amount of, for example, about 0.01 to about 3 weight percent, or about 0.05 to about 1 weight percent, or about 0.1 to about 0.5 weight percent based on the total weight of the oral care composition. In various embodiments, an active ingredient useful for pain relief can be present in an amount of less than about 2 weight percent, less than about 1 weight percent, or less than about 0.5 weight percent, based on the total weight of the oral care composition. In various embodiments, an active ingredient useful for pain relief can be present in an amount of at least about 0.01 weight percent, at least about 0.5 weight percent, at least about 1 weight percent, or at least about 2 weight percent, based on the total weight of the oral care composition.

In various embodiments, benzocaine can be included in the oral care composition to provide pain relief to the treatment area. The benzocaine can be present in an amount of, for example, about 5 to about 30, or about 10 to about 25 weight percent, or about 15 to about 20 weight percent, based on the total weight of the oral care composition. In various embodiments, an active ingredient useful for pain relief can be present in an amount of less than about 30 weight percent, less than about 20 weight percent, or less than about 10 weight percent, based on the total weight of the oral care composition. In various embodiments, an active ingredient useful for pain relief can be present in an amount of at least about 1 weight percent, at least about 5 weight percent, at least about 15 weight percent, or at least about 20 weight percent, based on the total weight of the oral care composition.

In various embodiments, the oral care composition can further comprise one or more additional ingredients. For example, the oral care composition can comprise at least one of a sweetener and a flavoring agent. In certain embodiments, the one or more sweeteners and/or flavoring agents can comprise sodium saccharin, monoammonium glycyrrhizate, a preferred flavor component, and combinations thereof.

In some embodiments, a sweetener can be present in an amount in the range of about 0.01 to about 5 weight percent, about 0.05 to about 2 weight percent, about 0.1 to about 1 weight percent, or about 0.2 to about 0.5 weight percent, based on the total weight of the oral care composition. In various embodiments, a sweetener can be present in an amount of at least about 0.01 weight percent, at least about 0.1 weight percent, or at least about 0.5 weight percent, based on the total weight of the oral care composition (e.g., with a maximum concentration of about 10 weight percent). In some embodiments, one or more flavoring agents can be present in an amount in the range of about 0.01 to about 10 weight percent, about 0.05 to about 5 weight percent, about 0.1 to about 2 weight percent, or about 0.5 to about 1 weight percent, based on the total weight of the oral care composition. The flavoring agent(s) can be present in an amount of at least about 0.01 weight percent, at least about 0.5 weight percent, at least about 1 weight percent, or at least about 2 weight percent, based on the total weigh of the oral care composition (e.g., with a maximum concentration of about 10 weight percent).

As described above, in various embodiments, the oral care composition is in the form of a cream. It is noted that a cream is typically differentiated from a gel by opacity. As described in the present disclosure, oral care compositions described herein can include opacifying agents in order to provide opaque creams. The inventive product provides a targeted and localized delivery of action for the duration of treatment, is soluble in oral mucosa. The formulated composition easily adheres to the tooth and gum, thereby, providing a maximum level of active ingredient (e.g., benzalkonium chloride) to be delivered at the treatment site to provide antiseptic activity and in certain embodiments, to further provide pain relief. The oral care compositions described herein can be manufactured in the form of a final oral care product having a desired dosage size, shape and weight via mixing and packing processes known in the art.

### EXAMPLES

### Example 1

An embodiment of an oral care composition (referred to as “Formula 1”) is provided. Table 1 below provides ingredients included in an embodiment of the oral care composition of the present disclosure.

Table 1 also includes the weight percentage of each ingredient, based on the total weight of the oral care composition. The primary function of each ingredient is also included.

TABLE 1
Cream Oral Care Composition - Formula 1

| | | Cream |
|---|---|---|
| Ingredient | Function | Wt. % |
| PEG-8 | Solubilizing Agent/Base | 30-50 |
| | Component | |
| PEG-75 | Structuring Agent | 20-40 |
| Sodium Saccharin | Flavoring Agent | 0.5-2 |
| Zinc Chloride (62% solution) | Active Ingredient | 0.05-0.4 |
| Sorbic Acid | Preservative | 0.01-0.5 |
| Benzocaine | Active Ingredient | 5-20 |
| Monoammonium Glycyrrhizate | Flavoring Agent | 0.5-2 |
| Flavor | Flavoring Agent | 0.5-2 |
| Chamomile | Antioxidant | 0.00-0.5 |
| Titanium Dioxide | Opacifying Agent | 0.05-0.5 |
| Poloxomer 407 | Structuring Agent | 1-3 |
| PEG/PPG-116/66 Copolymer | Structuring Agent | 1-6 |
| Benzalkonium Chloride (50% | Active Ingredient | 0.1-0.3 |
| solution) | (Antiseptic Agent) | |

It is noted that Formula 1 is in the form of a cream and further includes benzalkonium chloride which is active and provides antiseptic activity. Formula 1 does not include any substantial amounts of carboxymethyl cellulose, pectin, or petrolatum. Furthermore, it is found that Formula 1 does not readily separate when subjected to conditions that is may be exposed to throughout the 2 year shelf life typical of this class of formula. In other words, Formula 1 is considered to be stable according to industry standards.

### Example 2

Formula 1, according to Example 1 above, is tested for antiseptic activity. The following method is used to measure antiseptic activity of Formula 1.

First, samples of Formula 1 are prewarmed to 37° C. Next, 1.0 mL of serum, 1.0 mL of bacterial culture, and 8.0 mL of antiseptic test product (i.e., Formula 1) are combined and mixed. The mixture is incubated at 37° C. for 10 min. Next the entire mixture is diluted in a neutralization buffer. The number of surviving organisms per mL is determined after 10 mins of incubation by plate count method.

The antiseptic activity of Formula 1 is shown in Table 2 below.

TABLE 2
Antiseptic activity of Formula 1

| Organism | Initial Level | 10 mins | % Reduction |
|---|---|---|---|
| C. albicans 18804 | 2.9E5/g | 1.7E2/g | 99.9 |
| S. mutans 25175 | 2.5E5/g | 3.0E2/g | 99.9 |
| A. oris 19246 | 3.1E5 | 2.7E2/g | 99.9 |

As illustrated in Table 2 above, the oral care composition provided in Example 1 above (i.e., Formula 1) is exposed to at least about 2.9E5/g of C. albicans 18804 organisms, at least about 2.5E5/g S. mutans 25175 organisms, and at least about 3.1E5/g A. oris 19246 organisms, and has about 1.7E2/g of C. albicans 18804 organisms, about 3.0E2/g S. mutans 25175 organisms, and about 2.7E2/g A. oris 19246 organisms after subjecting the oral care composition to a temperature of about 37° C. for 10 mins. In other words, embodiments of the oral care compositions described herein demonstrate at least about a 99% reduction in bacteria counts for C. albicans 18804 organisms, S. mutans 25175 organisms, and A. oris 19246 organisms after subjecting the oral care composition to a temperature of about 37° C. for 10 mins.

### Example 3

A gel PEG-based formula, Formula 2, is prepared for comparison purposes. Table 3 below provides ingredients included in an embodiment of the oral care composition of the present disclosure. Table 3 also includes the weight percentage of each ingredient, based on the total weight of the oral care composition.

The primary function of each ingredient is also included.

TABLE 3
Gel Oral Care Composition - Formula 2

| | | Gel Formula 2 |
|---|---|---|
| Ingredient | Function | Wt % |
| PEG-8 | Base Component | 40-60 |
| PEG-75 | Structuring Agent | 20-40 |
| Sodium Saccharin | Flavoring Agent | 0.5-2 |
| Zinc Chloride | Active Ingredient | 0.00-0.5 |
| Sorbic Acid | Preservative | 0.01-0.5 |
| Benzocaine | Active Ingredient | 5-20 |
| Monoammonium Glycyrrhizinate | Flavoring Agent | 0.5-2 |
| Flavor | Flavoring Agent | 0.5-2 |
| Benzalkonium Chloride | Active Ingredient | 0.1-0.3 |
| | | 100.0000 |

The antiseptic activity of Formula 2 is shown in Table 4 below.

TABLE 4
Antiseptic activity of Formula 2

| Organism | Initial Level | 10 mins | % Reduction |
|---|---|---|---|
| C. albicans 18804 | 2.9E5/g | 4.5E2/g | 99.8 |
| S. mutans 25175 | 2.5E5/g | 2.7E2/g | 99.9 |
| A. oris 19246 | 3.1E5/g | 2.3E2/g | 99.9 |

Formula 2 is a comparative example demonstrating that antiseptic activity of a composition in other forms, such as the gel form of Formula 2. As discussed in detail above, the compositions of the present disclosure are provided in the form of a cream.

### Example 4

A cream petrolatum-based formula, Formula 3, is prepared for comparison purposes. Table 5 below provides ingredients included in an embodiment of the oral care composition of the present disclosure. Table 5 also includes the weight percentage of each ingredient, based on the total weight of the oral care composition. The primary function of each ingredient is also included.

TABLE 5
Cream, petrolatum-based Oral Care Composition - Formula 3

| | | Cream Formula 3 |
|---|---|---|
| Ingredient | Function | Wt % |
| PEG-8 | Solubilizer | 1-10 |
| Petrolatum | Structuring Agent | 30-60 |
| Sodium Saccharin | Flavoring Agent | 0.5-2 |
| Zinc Chloride | Active Ingredient | 0.00-0.5 |
| Pectin | Structuring Agent | 5-15 |
| Benzocaine | Active Ingredient | 5-20 |
| Cellulose Gum | Structuring Agent | 5-20 |
| Methyl Salicylate | Flavoring Agent | 0.1-2 |
| Benzalkonium Chloride | Active Ingredient | 0.1-0.3 |
| Gelatin | Structuring Agent | 5-20 |
| Mineral Oil | Solubilizer | 1-8 |
| | | 100.0000 |

The antiseptic activity of Formula 3 is shown in Table 6 below.

TABLE 6
Antiseptic activity of Formula 3

| Organism | Initial Level | 10 mins | % reduction |
|---|---|---|---|
| C. albicans 18804 | 9.5E5/g | 5.2E2/g | 99.9 |
| S. mutans 25175 | 8.8E5/g | 2.6E3/g | 99.7 |
| A. oris 19246 | 9.1E5/g | 5.3E4/g | 94.1 |

Antiseptic testing of Formula 3 has demonstrated that petrolatum-based formulas do not have sufficient antiseptic activity with benzalkonium chloride at about 0.1% after 10 min incubation to demonstrate a 99% reduction in each of C. albicans 18804 organisms, S. mutans 25175 organisms, and A. oris 19246 organisms. As such, the benzalkonium chloride in the petrolatum-based Formula 3 is not considered an active antiseptic agent according to the definition of an active antiseptic agent provided herein (i.e., capable of demonstrating at least a 99% reduction in all three bacteria types after an incubation period of 10 mins at 37° C.).

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. An oral care composition comprising:
a water-miscible base component;
an opacifying agent;
a structuring agent; and
an active antiseptic agent effective for providing antiseptic activity;
wherein the oral care composition is opaque and exhibits antiseptic activity.

2. The oral care composition of claim 1, wherein the oral care composition is substantially free of petrolatum.

3. The oral care composition of claim 1, wherein the oral care composition comprises petrolatum in an amount of less than 20% by weight, based on the total weight of the oral care composition.

4. The oral care composition of claim 1, wherein the water-miscible base comprises polyethylene glycol-8 (PEG-8).

5. The oral care composition of claim 4, wherein the PEG-8 is present in an amount of about 30 to about 50 wt. %, based on the total weight of the oral care composition.

6. The oral care composition of claim 1, wherein the opacifying agent is titanium dioxide.

7. The oral care composition of claim 1, wherein the opacifying agent is present in an amount of about 0.05 to about 0.5 wt. %, based on the total weight of the oral care composition.

8. The oral care composition of claim 1, wherein the structuring agent is selected from the group consisting of PEG/PPG-116/66 copolymer, poloxomer 407, PEG-75, and combinations thereof.

9. The oral care composition of claim 1, wherein the structuring agent is not negatively charged.

10. The oral care composition of claim 1, wherein the structuring agent is present in an amount of about 20 to about 50 wt. %, based on the total weight of the oral care composition.

11. The oral care composition of claim 1, wherein the active antiseptic agent comprises benzalkonium chloride.

12. The oral care composition of claim 11, wherein the benzalkonium chloride is present in an amount of less than about 1 wt. %, based on the total weight of the oral care composition.

13. The oral care composition of claim 1, wherein the oral care composition is in the form of a cream.

14. The oral care composition of claim 1, further comprising one or more additional active ingredients.

15. The oral care composition of claim 14, wherein the one or more additional active ingredients are selected from the group consisting of benzocaine, menthol, zinc chloride, and combinations thereof.

16. The oral care composition of claim 1, further comprising a flavoring agent.

17. The oral care composition of claim 16, wherein the flavoring agent is present in an amount in the range of about 0.1 to about 2 weight percent, based on the total weight of the oral care composition.

18. The oral care composition of claim 1, wherein the antiseptic activity exhibited by the oral care composition is defined in that the oral care composition is effective to cause at least a 99% reduction in bacteria counts for C. albicans 18804 organisms, S. mutans 25175 organisms, and A. oris 19246 organisms after contact between the oral care composition and the organisms at a temperature of about 37° C. for a time of about 10 mins.
